(19) United States
(12) Patent Application Publication
(10) Pub. No.: US 2004/0033961 A1
(43) Pub. Date: Feb. 19, 2004

(54) Therapeutic tannate compositions

(76) Inventors: Larry J. Gremminger, Richmond, TX (US); Robert S. Tutag, Edwards, CO (US)

Correspondence Address: BLACKWELL SANDERS PEPER MARTIN LLP, 720 OLIVE STREET, SUITE 2400, ST. LOUIS, MO, 63101, US

(21) Appl. No.: 10/064,805

(22) Filed: Aug. 19, 2002

(51) Int. Cl. A61K 31/7024

(52) U.S. Cl. 514/23

(57) ABSTRACT

The present invention relates to novel therapeutic compositions having pharmaceutically effective amounts of pseudoephedrine tannate and dexchlorpheniramine tannate, as well as, in one embodiment, a pharmaceutically effective amount of dextromethorphan tannate. The present invention also encompasses a method of using the novel therapeutic compositions, above, for symptomatically treating and relieving, among other things, the distress of coryza associated with sinusitis, allergic rhinitis, the common cold, and similar discrasias of the upper respiratory tract in affected patients.

### BACKGROUND OF INVENTION

The present invention relates generally to the field of antihistaminic/decongestant/antitussive tannate compositions. More specifically, the present invention relates to such tannate compositions having as essential active ingredients pseudoephedrine tannate and dexchlorpheniramine tannate. In some embodiments, the present invention relates to such tannate antihistamine compositions having the additional active ingredient dextromethorphan tannate.

Many tannic acids, a heterogeneous class of polymers with varying hydroxybenzenecarboxylic acids, occur in nature. In general usage, as in the present case, the terms tannic acid or tannin refer to gallotannic acid, the internal ester of gallic acid. The esters usually contain glucose, or less frequently, other sugars.

The color of tannic acid varies from yellowish-white to light brown. Physical forms of the acid vary from amorphous powder to glistening scales or spongy masses. It is very soluble in water, warm glycerol or alcohol.

Tannic acids occur in the bark and fruit of many plants, particularly the oak species, and are produced commercially from the Turkish and Chinese nutgall. Commercial preparations contain 5-10% water and have an average molecular weight of about 1700 daltons.

Pseudoephedrine, known chemically as α-[1-(methylamino)ethyl]-, [S-(R*,R*)]-Benzenemethanol, is a well-known sympathomimetic amine commonly used as the hydrochloride salt for its decongestant properties. Its nasopharyngeal vasoconstrictor and bronchodilator actions are about the same as those of ephedrine; however, it is weaker in its pressor, cardiac, mydriatic and central-stimulant actions.

Pseudoephedrine hydrochloride is prepared by acetylation of (−)-Ephedrine hydrochloride to produce (+)-Nacetylpseudoephedrine hydrochloride, which is then deacetylated to yield pseudoephedrine hydrochloride. It is available in the form of a fine, white crystalline powder having a faint, characteristic odor. Pseudoephedrine hydrochloride is very soluble in water and freely soluble in alcohol, and has a melting point between 182° and 185° C.

Dexchlorpheniramine, known chemically as γ-(4-Chlorophenyl)-N,N-dimethyl-2-pyridinepropanamine, is a widely used antihistamine, particularly in the form of its maleate salt. The latter is a white, odorless crystalline powder with a melting point between 113° and 115° C., and is soluble in water and alcohol. Dexchlorpheniramine maleate is produced by reacting equimolar portions of the base with maleic acid.

Dextromethorphan, or d-3-methoxy-N-methylmorphinan, is a synthetic morphine derivative prepared from morphinan, the parent substance of the morphine alkaloids. Its hydrobromide salt is used exclusively as an antitussive agent, which acts centrally to elevate the threshold for coughing. It is approximately equal in that function to codeine, but neither depresses the central nervous system nor shares the analgesic or addictive properties of codeine.

Compounds having antihistaminic, antitussive and/or decongestant properties in the form of either their free base or salts, e.g., hydrochloride, hydrobromide, maleate, citrate, tannate, etc., are well known. The tannate salts of antihistamines, antitussives and decongestants are especially desirable because they are generally stable and have few undesirable side effects.

The tannate salts of antihistaminic, antitussive and decongestant bases are traditionally prepared by reacting the free base with tannic acid in the presence of a volatile solvent such as isopropanol or ethanol. The reactants are maintained at 60°-70° C. with mixing for about one hour, allowed to cool to room temperature and then filtered, washed with cold solvent and dried under vacuum. Alternative procedures in which water is used as the solvent are described in U.S. Pat. No. 5,599,846 and U.S. Pat. No. 5,663,415.

### SUMMARY OF INVENTION

The present invention relates to novel therapeutic compositions having pharmaceutically effective amounts of pseudoephedrine tannate and dexchlorpheniramine tannate, as well as, in one embodiment, a pharmaceutically effective amount of dextromethorphan tannate. The present invention also encompasses a method of using the novel therapeutic compositions, above, for symptomatically treating and relieving, among other things, the distress of coryza associated with sinusitis, allergic rhinitis, the common cold, and similar discrasias of the upper respiratory tract in affected birds and mammals.

### DETAILED DESCRIPTION

It has now been found that novel pharmaceutical compositions having at least the active ingredients pseudoephedrine tannate and dexchlorpheniramine tannate have superior antihistaminic and decongestant properties compared with the use of either one of the tannate compounds alone. The present novel compositions also have antihistaminic and decongestant properties superior to other pseudoephedrine and/or dexchlorpheniramine salts either singly or in combination.

The compositions of the present invention may be prepared for oral administration in the form of powders, capsules, elixirs, syrups, and the preferred form of tablets or suspensions. The suspension is formulated such that each 5 mL dose of suspension would contain approximately 0.25 to 5.0 mg of dexchlorpheniramine tannate and 50 to 100 mg of pseudoephedrine tannate for the embodiment having only those two active ingredients. Example 1, which follows, illustrates a typical formulation of the present composition made using these two tannates and suitable carriers, coloring, natural and artificial flavoring, preservatives, sweeteners, and buffers.

### EXAMPLE 1

| INGREDIENT | Milligrams/5 mL Suspension |
|---|---|
| Pseudoephedrine Tannate | 75.0¹ |
| Dexchlorheniramine Tannate | 2.5¹ |
| Glycerin | 631.1 |
| Xanthan Gum | 15.0 |
| Methylparaben | 5.0 |
| Magnesium Aluminum Silicate | 37.5 |
| Citric Acid | 10.0 |
| Sodium Benzoate | 5.0 |
| Sodium Citrate | 5.0 |
| Sodium Saccharin | 250 |
| Sucrose | 2250.0 |
| Strawberr Flavor | 172.5 |
| Banana Flavor | |
| FD&C Red #40 | 1.0 |
| Purified Water, USP | Adjust to 5.0 mL |

¹5% excess added during manufacturing

In an alternative embodiment of the present invention having the addition of dextromethorphan tannate, the suspension is formulated such that each 5 mL dose of suspension would contain approximately 0.25 to 5.0 mg of dexchlorpheniramine tannate, 15 to 40 mg of dextromethorphan tannate, and 50 to 100 mg of pseudoephedrine tannate. Example 2, which follows, illustrates a typical formulation of such an alternative embodiment of the present composition made using these two tannates and suitable carriers, coloring, natural and artificial flavoring, preservatives, sweeteners, and buffers.

### EXAMPLE 2

| INGREDIENT | Milligrams/5 mL Suspension |
|---|---|
| Pseudoephedrine Tannate | 75.0¹ |
| Dextromethorphan Tannate | 25.0¹ |
| Dexchlorpheniramine Tannate | 2.5¹ |
| Glycerin | 631.1 |
| Xanthan Gum | 15.0 |
| Methylparaben | 5.0 |
| Magnesium Aluminum Silicate | 37.5 |
| Citric Acid | 10.0 |
| Sodium Benzoate | 5.0 |
| Sodium Citrate | 5.0 |
| Sodium Saccharin | 25.0 |
| Sucrose | 2250.0 |
| Cotton Candy Flavor | 5.2 |
| FD&C Blue #1 | 1.7 |
| Purified Water, USP | Adjust to 5.0 mL |

¹5% excess added during manufacturing

The above formulations may be modified by additives and adjuvants without departing from the scope of the present invention. For example, the specific flavoring and color used in the formulations may vary widely in order to produce products with a broad range of appeal to those who use such products. Similarly, various carriers, sweeteners, preservatives, and buffers shown above may also be replaced by other such compounds, such as excipients, etc., that are suitable for the purposes of the present invention. The key to the present invention lies in the novel combination of tannates set forth in the examples above, and not in the specific identity of these secondary compounds.

The compositions of the present invention may be used for symptomatically treating and relieving distress of coryza associated with sinusitis, allergic rhinitis, the common cold, and similar discrasias of the upper respiratory tract in affected patients. It is to be understood, however, that the use of the novel compositions of the present invention are not limited to treatment of the conditions enumerated above, and that other uses of the present novel compositions will be readily apparent to those of skill in the art.

The dosage administered to a specific mammal or bird will be dependent on the weight, age, and health of the recipient, the existence of and kinds of concurrent treatment, and the degree and type of effect desired. Determining appropriate dosages with respect to the compositions of the present invention is well within the skill of those practicing in the medical and veterinary arts. The teachings of the present invention simply require the use of a pharmaceutically effective amount of the active ingredients of the present invention. For dexchlorpheniramine, a pharmaceutically effective amount is generally considered to be about 0.25 to 10 mg; for pseudoephedrine 25 to 150 mg; and for dextromethorphan 10 to 75 mg. Depending on the particular application, it may be possible to depart somewhat from these measurements and still achieve a pharmaceutically desired result.

It is understood that the above formulations are examples of formulations produced in accordance with the teachings of the present invention and do not represent all possible formulations that could produced in accordance with the teachings of the present invention. Is is contemplated that, based upon the present disclosure, skilled practitioners in the art may realize numerous variations that do not depart from the spirit and scope of the present invention. It is intended that a lattitude of modification, substitution, and change can be employed in the compositions and methods disclosed in the claims, and that some features of the invention may be used without the corresponding use of others.

1. A therapeutic pharmaceutical composition comprising
a) a pharmaceutically effective amount of pseudoephedrine tannate; and
b) a pharmaceutically effective amount of dexchlorpheniramine tannate:

2. The therapeutic pharmaceutical composition of claim 1, further comprising a pharmaceutically effective amount of dextromethorphan tannate.

3. The therapeutic pharmaceutical composition of claim 1 wherein said therapeutic pharmaceutical composition is in liquid suspension form.

4. The therapeutic pharmaceutical composition of claim 1 wherein said therapeutic pharmaceutical composition is in tablet form.

5. The therapeutic pharmaceutical composition of claim 3 wherein the pharmaceutically effective amount of pseudoephedrine tannate is from about 50 mg to about 100 mg per 5 mL volume of suspension.

6. The therapeutic composition of claim 3 wherein the pharmaceutically effective amount of dexchlorpheniramine tannate is from about 0.25 mg to 5.0 mg per 5 mL volume of suspension.

7. The therapeutic composition of claim 2 wherein the pharmaceutically effective amount of dextromethorphan tannate is from about 15 to about 40 mg per 5 mL volume of suspension.

8. The therapeutic pharmaceutical composition of claim 5 wherein the pharmaceutically effective amount of pseudoephedrine tannate is about 75 mg per 5 mL volume of suspension.

9. The therapeutic pharmaceutical composition of claim 6 wherein the pharmaceutically effective amount of dexchlorpheniramine tannate is about 2.5 mg per S mL volume of suspension.

10. The therapeutic pharmaceutical composition of claim 7 wherein the pharmaceutically effective amount of dextromethorphan tannate is about 25 mg per 5 mL volume of suspension.

11. A therapeutic pharmaceutical composition comprising
a) about 75 mg of pseudoephedrine tannate; and
b) about 2.5 mg of dexchlorpheniramine tannate.

12. A therapeutic pharmaceutical composition comprising
a) about 75 mg of pseudoephedrine tannate;
b) about 2.5 mg of dexchlorpheniramine tannate; and
c) about 25 mg of dextromethorphan tannate.

13. The therapeutic pharmaceutical composition of either of claims 11 or 12 further comprising suitable pharmaceutical carriers.

14. A method for symptomatically treating and relieving the distress of coryza associated with sinusitis, allergic rhinitis, the common cold, and similar dyscrasias of the upper respiratory tract in affected patients comprising oral administration of a therapeutic amount of a composition as disclosed in claim 11 to patients in need of such treatment.

15. A method for symptomatically treating and relieving the distress of coryza associated with sinusitis, allergic rhinitis, the common cold, and similar dyscrasias of the upper respiratory tract in affected patients comprising oral administration of a therapeutic amount of a composition as disclosed in claim 12 to mammals and birds in need of such treatment.
